(19) United States
(12) Patent Application Publication
(10) Pub. No.: US 2021/0049930 A1
(43) Pub. Date: Feb. 18, 2021

(54) MEDICAMENT DISPENSER SIMULATOR

(71) Applicant: SHORE PRODUCT GROUP LIMITED, Leith, Edinburgh Lothian (GB)

(72) Inventors: Iain Robert MORTIMER, Edinburgh Lothian (GB); Ian SCRIMGEOUR, Dunbar, East Lothian (GB); James Donald MCLUSKY, Edinburgh Lothian (GB); Nicholas FOLEY, Edinburgh Lothian (GB); William Jeremy DAVIES, Edinburgh Lothian (GB)

(73) Assignee: SHORE PRODUCT GROUP LIMITED, Leith, Edinburgh Lothian (GB)

(21) Appl. No.: 16/964,039

(22) PCT Filed: Jan. 22, 2019
(86) PCT No.: PCT/GB2019/050167

§ 371 Date: Jul. 22, 2020

(30) Foreign Application Priority Data
Jan. 22, 2018 (GB) 1801006.6
Oct. 5, 2018 (GB) 1816284.2

(51) Int. Cl. G09B 23/28; A61M 11/00

(52) U.S. Cl. CPC G09B 23/28; A61M 11/007; A61M 2210/0618; A61M 2205/103; A61M 2205/073

(57) ABSTRACT

A device (10) for simulating the dispensing of a medication into the nasal passage. The device includes a device body (12), the device body defining an extending portion (14) for inserting into the nasal passage of a user. An activation mechanism (16) movable from a start position to a dispensed position can be returned to the start position by manipulation of part of the device. Manipulation may be by rotation of a main part of the device body (12) relative to the extending portion (14).

### FIELD

The present invention relates to the field of devices which simulate the dispensing of a medicament, in particular, to devices which simulate the nasal dispensing of medicaments.

### BACKGROUND

Recent advances in medicine have revealed that medication can be delivered up the nasal passage of patient. Some pharmaceutical companies are supplying drugs in this form for a patient to take at home.

One particular type of nasal dispenser requires part of the dispenser to be inserted up the patient's nose and press a button or plunger to release the medication.

There are different types of nasal dispenser, In one exemplary type of device, full delivery of the medication is a three-stage process. The first stage is to compress air within the device, the second stage is to release an obstruction within the dispenser, allowing the compressed air to rush up the nose carrying the medication and third stage is to indicate to the patient that the dispensing of the medication is complete.

As the plunger passes through each of these stages, the resistance felt by the patient changes and it is only through use that the patient develops the correct technique for dispensing the medication into the nasal passage. However, during this initial trial and error stage, there is a risk the patient under doses on the medication to the detriment of their health.

Simulators are known, particularly in the fields of inhalation of a medication or the needle injection of a medication. The purpose of a simulator is to allow the patient to repeatedly practice the action of delivering medication prior to using the actual medication delivery device. To achieve the repeatability aspect of a simulator requires the device to be reset. In the symbolises this usually is achieved by use of a tool or by an additional mechanism on the device.

The drawback with additional tooling or mechanisms is they detract from the object of a simulator which is to replicate a medication dispenser in both operation and appearance.

### SUMMARY

According to a first aspect of the invention there is provided a device for simulating the dispensing of a medication into the nasal passage, the device comprising:

a device body, the device body defining an extending portion for inserting into the nasal passage of a user; and

an activation mechanism movable from a start position to a dispensed position;

wherein the activation mechanism can be returned to the start position by manipulation of part of the device.

In at least one embodiment of the invention, providing a device for simulating the dispensing of medication into the nasal passage which can be reset by manipulation of part of the device provides a device which visually appears identical to the device which is a simulating and can be reset intuitively without the need for additional tools.

The activation mechanism may be movable from the start position to the dispensed position by the application of a force.

The activation mechanism may be moveable relative to the device body when moving from the start position to the dispensed position.

The activation mechanism may be in the telescopic relationship with the device body.

In a typical real device for dispensing a medication into the nasal passage the activation mechanism comprises a button on the device body, distal to the extending portion (nozzle) that is inserted into a nasal passage. The user pushing the button telescopically inside the body (typically linearly towards the nozzle) causes dispensing of the medicament. The simulating devices of the invention can have a telescopic button/device body arrangement that can have a similar appearance and behaviour as that of a corresponding real device.

The activation mechanism may engage an at least one activation profile when moving from the start position to the dispensed position.

The at least one activation profile may be defined by a guide member located within the device body.

Alternatively or additionally the at least one activation profile may be defined by an internal surface of the device body.

The activation mechanism may include an at least one engaging member, the/each engaging member adapted to engage with the/one of the activation profiles. The engaging member or members may comprise a projection or projections (or a depression or depressions) directed radially outwards or radially inwards of a component of the activation mechanism, such as the body of a button. The projections or depressions can interact with the activation profile as the activation mechanism is moved.

Alternatively, or additionally the at least one activation profile may be provided on a surface of the activation mechanism, for example on the surface of a button. Where the at least one activation profile is provided on a surface of the activation mechanism then an engaging member or members may be provided on the device body, or on a guide member within the body to engage with the profile.

The/each activation profile may resist movement of the activation mechanism from the start position to the dispensed position.

The activation profile can thus allow accurate mimicking of the behaviour of a real device. For example, as a user moves an activation device (such as a button) on a real device, they experience changes in the force required, the speed at which the button can move, hear sound and may feel vibration. The activation profile on a simulating device of the invention can be arranged to provide similar, close to identical or even identical haptic and audio feedback to the user.

One or both of the/each activation profile or/and engaging member may move in a direction perpendicular to the direction of travel of the activation mechanism as the activation mechanism moves from the start position to the dispensed position.

In one embodiment the/each activation profile defines a raised profile which causes the activation mechanism engaging member to deflect in a direction perpendicular to the direction of travel of the activation mechanism.

More generally an engaging member or engaging members engage with an activation profile as the activation mechanism is moved.

In the dispensed position, the activation mechanism engaging member may be prevented from returning to the start position along the activation profile.

Engagement between the activation mechanism engaging member and activation profile may be such that movement from the dispensed position back to the start position along the activation profile is prevented.

For example, the activation mechanism engaging member, or an end of the engaging member may latch into a feature of the activation profile such as a depression or a groove. Latching may include, for example, an end of an activation mechanism engaging member hooking over a step in the activation profile. Alternatively an end of an activation mechanism engaging member fitting into a depression on an activation profile.

The device may include a biasing mechanism the biasing mechanism biasing the activation mechanism to the start position.

The biasing mechanism may bias the activation mechanism to the start position in a linear and/or rotational direction

The biasing mechanism may be a spring.

The spring may be a compression spring, movement from the start position to the dispensed position compressing the spring.

In the dispensed position, manipulation of part of the device may move the/each activation mechanism engagement member from the dispensed position to a disengaged position, in the disengaged position the/each activation mechanism engage member being disengaged from the/each activation profile.

The part of the device, manipulation of which returns the activation mechanism to the start position may be the device body.

Alternatively or additionally the part of the device which returns the activation mechanism to the start position may be the activation mechanism (e.g. a button of an activation mechanism. As long as the part of the device which is manipulated is common to the external appearance of both the device for simulating the dispensing medication and the actual medication dispensing device itself, then a realistic user experience both from a visual and an operative perspective can be achieved.

The activation mechanism may move from the disengaged position to the start position upon application of force.

The force may be applied by the biasing mechanism.

The activation mechanism may be rotated from the dispensed position to the disengaged position.

Where the biasing mechanism is a spring, the spring may be a torsional spring as well as or instead of a compression spring.

In a convenient embodiment the activation mechanism comprises a button on the device body, distal to the extending portion (nozzle) that is inserted into a nasal passage. The user pushing the button telescopically inside the body, against the biasing of a compression spring (and typically linearly towards the nozzle) causes simulation of dispensing a medicament.

In such an embodiment the body of the device may comprise two parts, one comprising the extending portion (nozzle) and the other a main body part in telescopic relationship with the button. Rotation is permitted between the nozzle part and the main body part. In this embodiment the button may be rotationally locked to the main body, but axial motion is allowed. e.g. by a key and groove or a key and splines arrangement between button and body. After moving the activation mechanism (button) axially to the dispensed position, it is prevented from returning to the start position by latching of an activation mechanism engaging member of the button to the activation profile in a manner such as described above. Rotating the main body relative to the nozzle part rotates the button against torsional bias provided by the compression spring to a position where the button is unlatched from the activation profile (i.e. the engaging member unlatches from the activation profile) allowing the button to return axially towards the start position. The button (and main body part) also rotates fully back to the start position by the action of the torsional bias in the compression spring.

As an alternative in such an embodiment, the button may rotationally lock to the nozzle part. For example, the nozzle part of the body may include a projection into the main body part that cooperates with the button, at least after it has started moving towards the dispensed position. The cooperation may be a key and groove arrangement or a key and splines arrangement. At the dispensed position the rotation of the nozzle part relative to the main body part can release the latching to the activation profile, allowing return of the button to the start position.

The device may further comprise a damping mechanism. A damping mechanism may be provided to provide further resistance to the movement of the activation mechanism from the start position to the dispensed position.

In some embodiments, the damper may be a linear damper.

Alternatively, the damper may be a rotary damper. For example a rotary damper making use of a damper member rotating in a fluid to provide a braking effect.

Where the damping mechanism is a linear damper, it may comprise a spring, e.g. a compression spring. In some embodiments the compression spring of the damper may be the biasing mechanism, the biasing mechanism biasing the activation mechanism to the start position.

According to a second aspect of the invention there is provided a device for simulating the dispensing of a medication into the nasal passage, the device comprising:

a device body, the device body defining an extending portion for inserting into the nasal passage of a user; and

an activation mechanism movable from a start position to a dispensed position; and

a biasing mechanism that biases the activation mechanism to the start position.

In devices according to the second aspect of the invention the activation mechanism may return to the start position by the action of the biasing mechanism without requiring manipulation of part of the device. Alternatively, a device according to the second aspect may require manipulation to return from the dispensed position to the start position.

The components and mechanisms discussed above with respect to the first aspect of the invention may be employed with devices according to the second aspect of the invention. For example, the use of activation profiles and engaging members to provide haptic and audio feedback. For further example where devices of the second aspect require manipulation to return to the start position the mechanisms including rotational locking of the activation member (button) to either a main body part or a nozzle body part of the device discussed above.

In an embodiment according to the second aspect, the activation mechanism comprises a button on the device body, distal to the extending portion (nozzle) that is inserted into a nasal passage. The user pushing the button telescopically inside the body, against the biasing of a compression spring (and typically linearly towards the nozzle) causes simulation of dispensing a medicament. On release of the button the button returns to the start position by the action of the compression spring. In such an embodiment a guide member maybe fitted inside the body and include at least one engaging member for engaging with an activation profile on a surface of the button. For example, the guide member or members may include one or more projections extending radially inwards to engage with an activation profile on the outer surface of the button. The activation profile may include a groove into which the projections fit when the button is in the start position, latching the button (e.g. by a hook feature) against the biasing action of the compression spring. This latching prevents the button from moving further away from the body than desired. but allows (by deflection of the projections) the button to be pushed into the body.

### BRIEF DESCRIPTION OF THE DRAWINGS

Embodiments of the present invention will now be described with reference to the accompanying drawings in which:

FIG. 1 is a side view of a device for simulating the dispensing of a medication into the nasal passage according to a first embodiment of the present invention;

FIG. 2 is a section through the device of FIG. 1;

FIG. 3 is an exploded perspective view of the device of FIG. 1;

FIG. 4 is a section through the device of FIG. 1 in the start position;

FIG. 5 is a section through the device of FIG. 1 in the dispensed position;

FIG. 6 is an exploded perspective view of a device for simulating the dispensing of a medication into the nasal passage according to a second embodiment of the present invention;

FIG. 7 shows in exploded perspective view another device for simulating the dispensing of a medication into the nasal passage according to another embodiment of the present invention; and

FIGS. 8A, 8B and 8C show a device for simulating the dispensing of a medication into the nasal passage according to a second aspect of the invention.

### DETAILED DESCRIPTION OF THE DRAWINGS

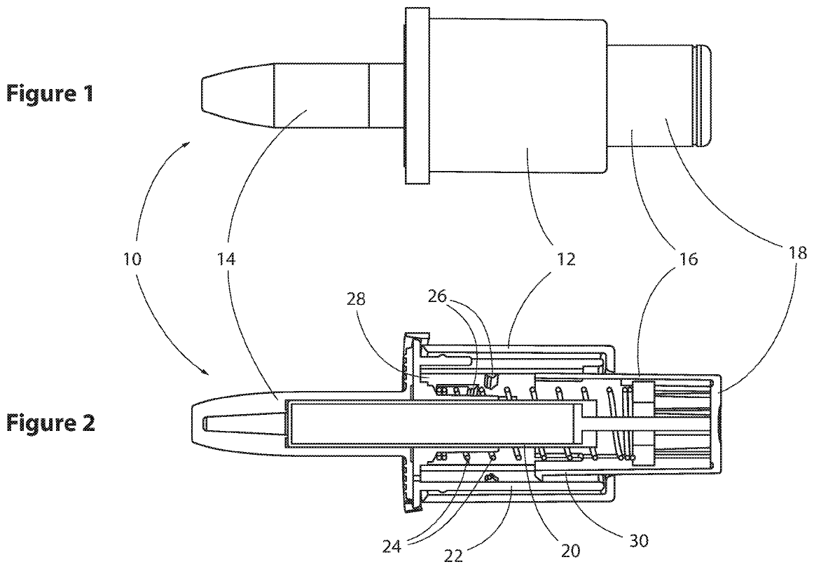

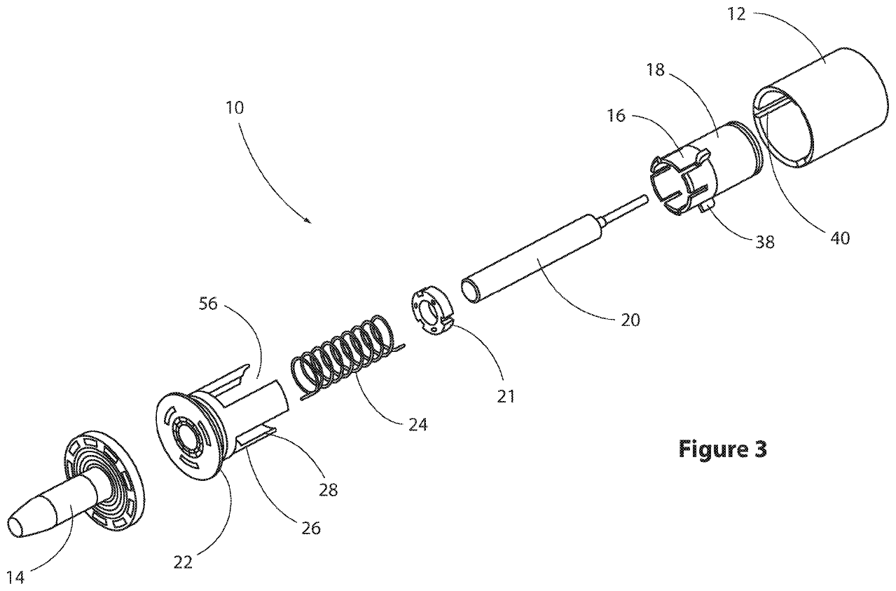

Referring to FIG. 1, a side view of a device 10 for simulating the dispensing of a medication into the nasal passage according to a first embodiment of the present invention, FIG. 2, a section through the device 10 of FIG. 1, and FIG. 3, an exploded perspective view of the device 10 of FIG. 1, the device 10 comprises a device body 12, the device body 12 defining an extending portion 14 (a nozzle) for inserting into the nasal passage of a user (not shown). The main part of the device body 12 and extending portion 14 can rotate one relative to the other. The device 10 further includes an activation mechanism 16 in the form of an activation button 18.

Referring particularly to FIGS. 2 and 3, the device 10 further includes a linear damper 20, a support ring 21, a guide member 22 and a biasing spring 24. The guide member 22 is in a fixed relationship to the extending portion 14.

Most clearly seen on FIG. 2, the device 10 further includes an activation profile 26 defined by a guide member internal surface 28. As will be described in more detail, during operation, the activation button 18 travels from the position shown in FIG. 2 (the start position) towards the extending portion 14, the activation button 18 including a number of engaging members 30 which engages with the activation profile 26 to provide haptic and audio feedback to the user as they operate the device 10.

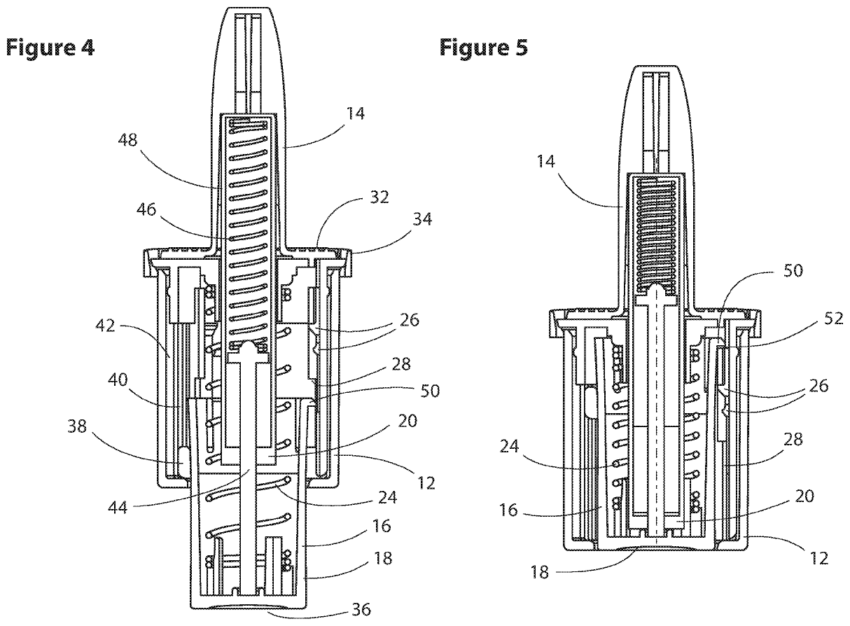

Operation of the device will now be described with reference to FIGS. 4 and 5, sections of the device of FIG. 1 in the start position (FIG. 4) and the dispensed position (FIG. 5) together with FIG. 3.

The user places the extending portion 14 between the tips of their forefinger and middle finger such that the fingers engage the upper side 32 of a body flange 34 and their thumb against a button end 36. The user then inserts the extending portion 14 into their nose and applies pressure to the activation button 18 which causes the activation button 18 to move towards the extending portion 14.

The activation button 18 defines an external key 38 which runs in a track 40 defined by a device body internal surface 42. This track 40 is linear and prevents the activation button 18 from deviating from a linear path as it travels from the start position towards the dispensed position. The movement of the activation button 18 is resisted by the damper 20. The damper 20 consists of a piston 44 which acts to compress a spring 46 contained within a damper housing 48. The damper 20 resists movement of the activation button 18 and is used, in this case, to simulate the compression of air within an actual medication dispenser.

As the activation button 18 travels down the body 12 it comes into contact with the guide member 22, particularly an external lip 50 defined by the activation button engaging members 30 comes into contact with the guide member activation profile 26. The profile 26 further resists movement of the activation button 18 and the engagement member lips 50 have to deflect inwardly, perpendicular to the direction of travel of the activation button 18, to travel over the profile 26. Each of the peaks on the profile 26 simulate an increased pressure required to trigger part of the process of delivering a medication through an actual medication dispenser.

As shown in FIG. 5 the activation button has reached the dispensed position and the lips 50 have come to rest against the back of the final peak 52 of the profile 26. This engagement prevents the activation button 18 returning to the start position under the action of the spring 24 which has been compressed during the transit of the activation button 18.

To return to the start position the user applies a rotational force to the device body 12, which due to the engagement between the activation button external key 38 and the device body track 40, causes the activation button 18 to rotate, moving the engaging member lips 50 to the side of the activation profile 26 and into a space 56 between each activation profile (best seen in FIG. 3). The rotation also applies to spring 14 acting against its torsional biasing. The user then releases the activation button 18 which travels back to the start position as the spring 24 releases the compression energy and torsional energy which has been applied to it.

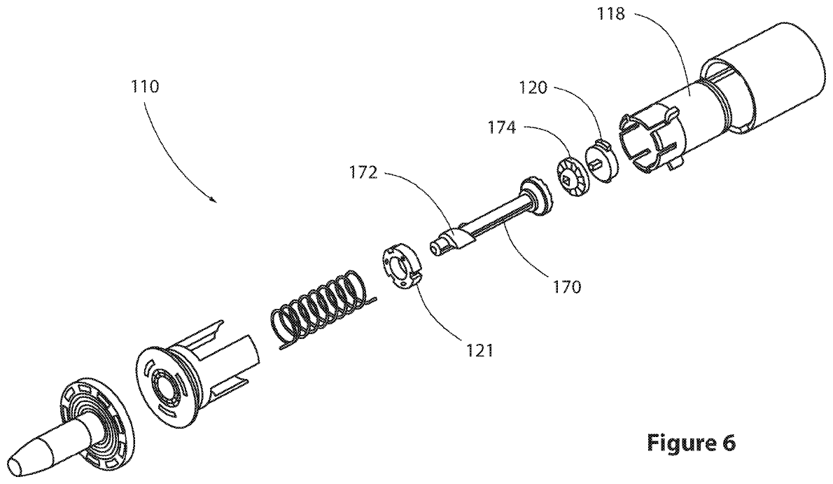

Reference is now made to FIG. 6, an exploded perspective view of a device, 110, for simulating the dispensing of a medication into the nasal passage according to a second embodiment of the present invention.

The device 110 of this second embodiment, differs from the device 10 of the first embodiment in that the device 110 incorporates a shaft 170 which defines an end profile 172. As the activation button 118 is pressed, the shaft 170 and the profile 172 passes through a complementary profile in the support ring 121 which causes rotation of the shaft 170. The shaft 170 is connected to a rotary damper 120 by means of a clutch 174. In this case the movement of the activation button 118 is resisted by the rotary damper resisting rotation of the shaft 170. The device 110 is reset in the same way as the device 10 of the first embodiment when the button 118 is released the clutch 174 disengages from the shaft 170 permitting the shaft to rotate back through the support ring 121.

It will be appreciated that such an arrangement allows for a simulator to be constructed which, as far as the user is concerned, operates and appears to be identical to the dispenser which the devices is simulating, thereby assisting in the successful use of the actual medication dispenser.

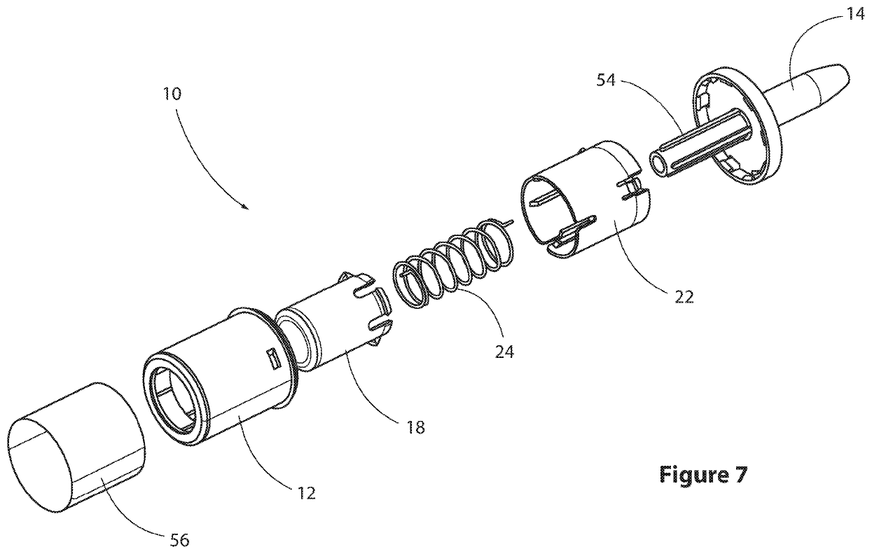

FIG. 7 shows in exploded perspective view a device similar to that shown in FIGS. 1 to 5 and with like parts numbered the same. A label 56 fits around body 12 in this example. In this example the extending portion 14 of body 12 incudes a projection 54 which extends into the body 12 when the device is assembled. Projection 54 carries splines 55. In use, as the button 18 is pressed towards extending portion (nozzle) 14, the splines engage key features on the inside surface of button 18 (not visible), rotationally locking the button to the extending portion, but allowing axial motion. Thus, when the device 10 of FIG. 7 is locked in the dispensed position, (in a manner similar to that of the device shown in FIG. 5), rotation of the nozzle 14 rotates button 18 relative to the body 12 and guide member 22. This releases the engagement between engaging members on the button 18 and the corresponding activation profile on the guide member 22, allowing a return of button 18 to the start position driven by the biasing forces (compression and torsional energy) provided by spring 24.

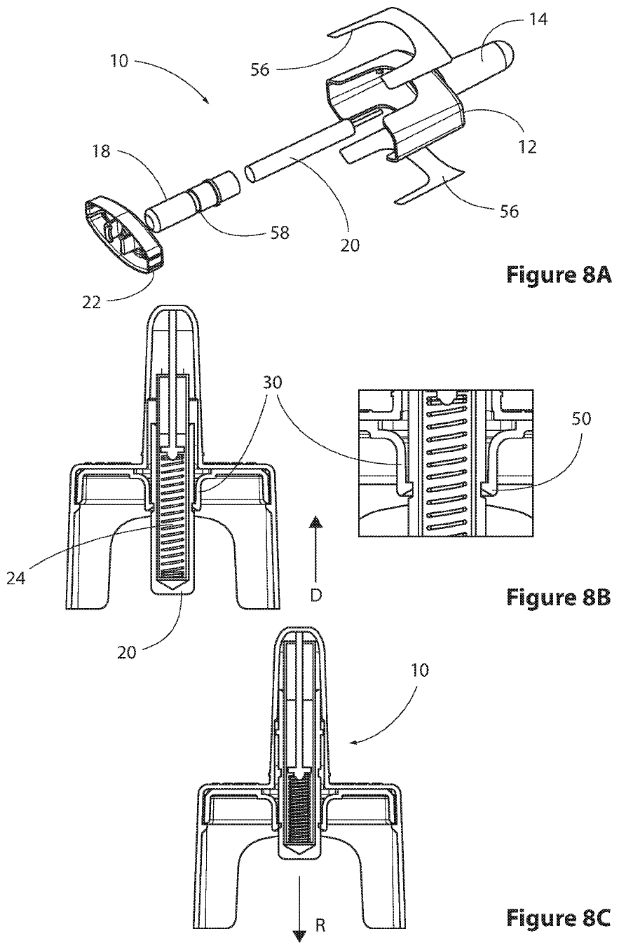

FIGS. 8A to 8C illustrate a device 10 in accordance with the second aspect of the invention. In exploded view FIG. 8A guide member 22, activating mechanism (button 18), linear damper 20, body 12 extending portion 14 and labels 56 are seen. Button 18 has on its external surface a circumferentially extending groove 58. The external surface of button 18 and the groove 58 constitute an activation profile for this device. As can be seen more clearly in cross section FIG. 8B and the accompanying magnified detail, the guide member 22 includes engaging members 30 in the form of projections having lips 50. Lips 50 fit into groove 58 on button 18. On pressing button 18 in direction D, the compression spring 24 of damper 20 resists, as does the positioning of lips 50 in groove 58. When sufficient pressure is applied, the lips 50 are forced out of groove 58 with an audible click. The button 18 can continue in direction D, and against the biasing provided by spring 24, to the dispensed position shown in FIG. 8C. On release, the button 18 returns to the start position as suggested by arrow R. The reengagement of the lips 50 in groove 58 another audible click is produced, mimicking the use of a real device.

1. A device for simulating the dispensing of a medication into the nasal passage, the device comprising:
a device body, the device body defining an extending portion for inserting into the nasal passage of a user; and
an activation mechanism movable from a start position to a dispensed position;
wherein the activation mechanism can be returned to the start position by manipulation of part of the device.

2. The device of claim 1, wherein the activation mechanism is movable from the start position to the dispensed position by the application of a force.

3. The device of claim 1, wherein movement of the activation mechanism is relative to the device body when moving from the start position to the dispensed position.

4. The device of claim 1, wherein the activation mechanism is in a telescopic relationship with the device body.

5. The device of claim 1, wherein the activation mechanism engages an at least one activation profile when moving from the start position to the dispensed position.

6. The device of claim 5, wherein the at least one activation profile is defined by a guide member located within the device body.

7. The device of claim 5, wherein the at least one activation profile is defined by an internal surface of the device body.

8. The device of claim 5, wherein the activation mechanism includes an at least one engaging member, the/each engaging member adapted to engage with the/one of the activation profiles.

9. The device of claim 5, wherein the/each activation profile resists movement of the activation mechanism from the start position to the dispensed position.

10. The device of claim 8, wherein one or both of the/each activation profile or/and engaging member moves in a direction perpendicular to the direction of travel of the activation mechanism as the activation mechanism moves from the start position to the dispensed position.

11. The device of claim 10, wherein the/each activation profile defines a raised profile which causes the activation mechanism engaging member to deflect in a direction perpendicular to the direction of travel of the activation mechanism.

12. The device of claim 8, wherein in the dispensed position, the activation mechanism engaging member is prevented from returning to the start position along the activation profile.

13. The device of claim 8, wherein the device includes a biasing mechanism, the biasing mechanism biasing the activation mechanism to the start position.

14. The device of claim 13, wherein the biasing mechanism biases the activation mechanism to the start position in a linear and/or rotational direction.

15. The device of claim 13, wherein the biasing mechanism is a spring.

16. The device of claim 15, wherein the spring is a compression spring, movement from the start position to the dispensed position compressing the spring.

17. The device of claim 15, wherein the spring is a torsional spring.

18. The device of claim 8, wherein, in the dispensed position, manipulation of a part of the device moves the/each activation mechanism engagement member from the dispensed position to a disengaged position, in the disengaged position the/each activation mechanism engage member being disengaged from the/each activation profile.

19. The device of claim 18, wherein the part of the device is the device body.

20. The device of claim 18, wherein the part of the device is the activation mechanism.

21. The device of claim 1, wherein the activation mechanism moves from the disengaged position to the start position upon application of force.

22. The device of claim 21, wherein the device includes a biasing mechanism, the biasing mechanism biasing the activation mechanism to the start position; and
wherein the force is applied by the biasing mechanism.

23. The device of claim 21, wherein the activation mechanism is rotated from the dispensed position to the disengaged position.

24. The device of claim 1, wherein the device further comprises a damping mechanism.

25. The device of claim 24, wherein the damper is a linear damper.

26. The device of claim 25, wherein the damper is a rotary damper.

27. A device for simulating the dispensing of a medication into the nasal passage, the device comprising:
a device body, the device body defining an extending portion for inserting into the nasal passage of a user; and
an activation mechanism movable from a start position to a dispensed position; and
a biasing mechanism that biases the activation mechanism to the start position.
